(19) United States
(12) Patent Application Publication
(10) Pub. No.: US 2010/0189717 A1
(43) Pub. Date: Jul. 29, 2010

(54) Plant Recombinant Human CTLA4IG and a Method for Producing the Same

(75) Inventors: Sang-Lin Kim, Seoul (KR); Hyun-Kwang Tan, Seoul (KR); Sang-Min Lim, Incheon-si (KR); Wuk-Sang Ryu, Gyeonggi-do (KR); Hahn-Sun Jung, Gyeonggi-do (KR); Song-Jae Lee, Seoul (KR); Cheon-Ik Park, Gyeonggi-do (KR); Seung-Hoon Kang, Gyeonggi-do (KR); Dong Il Kim, Incheon-si (KR)

Correspondence Address: DORITY & MANNING, P.A., POST OFFICE BOX 1449, GREENVILLE, SC, 29602-1449, US

(73) Assignee: Boryung Pharmaceutical Co., Ltd., Seoul (KR)

(21) Appl. No.: 12/668,766

(22) PCT Filed: Jul. 9, 2008
(86) PCT No.: PCT/KR2008/004029

§ 371 Date: Jan. 12, 2010

(30) Foreign Application Priority Data
Jul. 26, 2007 (KR) 10-2007-0074945

(51) Int. Cl. A61K 39/395; C12N 15/74; C12N 5/10; C12P 21/00; C07K 16/18; A61P 37/04

(52) U.S. Cl. 424/133.1; 435/320.1; 435/419; 435/69.6; 530/387.3

(57) ABSTRACT

The present invention provides a recombinant vector pBI-3D-hGalT or pBI-35S-hGalT containing a human β1,4-galactosyltransferase gene; a cell line transformed with a recombinant vector pMYN414 containing a cytotoxic T-lymphocyte anti-gen A-immunoglobulin (CTLA4Ig) fusion protein gene and the recombinant vector pBI-3D-hGalT or pBI-355-hGalT; and a method for producing a plant-derived recombinant human CTLA4Ig (CTLA4Igp) fusion protein with a human glycan structure using the same. The plant cell-derived recombinant human CTLA4Ig fusion protein (CTLA4Igp), which has a human glycan structure and is produced according to the present invention, exhibits an improved in vivo half life as compared to conventional plant-derived proteins, due to the presence of a human-like glycan structure. Consequently, the present invention using the plant expression system enables low-cost mass production of a CTLA4Igp fusion protein having an immunosuppressive activity comparable to that of the CTLA4IgM fusion protein expressed in conventional animal cell expression systems.

### TECHNICAL FIELD

The present invention relates to a recombinant vector pBI-3D-hGalT or pBI-35S-hGalT containing a human β1,4-galactosyltransferase gene; a cell line transformed with a recombinant vector pMYN414 containing a cytotoxic T-lymphocyte antigen 4-immunoglobulin (CTLA4Ig) fusion protein gene and the recombinant vector pBI-3D-hGalT or pBI-35S-hGalT; and a method for producing a plant-derived recombinant human CTLA4Ig (CTLA4Ig^{P}) fusion protein with a human glycan structure using the same.

### BACKGROUND ART

Immunomodulators may be broadly classified into immunoenhancers and immunosuppressants, depending on their pharmacological action augmenting or suppressing immune functions. Among these substances, immunosuppressants are receiving a great deal of attention for their importance in organ transplantation due to an urgent need of drugs to prevent transplant rejection with a recent rapid increase of organ transplantation surgery, for example, heart, liver and kidney transplantation. These drugs are also attracting a great deal of interest because commercial attention has been directed to development of therapeutic drugs for the treatment of autoimmune diseases that are inflammatory diseases caused by hyperfunction of the immune system daring to attack the body's own tissues.

T-cell activation plays an important role in triggering of transplantation rejection. Two types of signals are required for full activation of T cells. The first signal is intracellular delivery of an activation signal by the interaction of the Major Histocompatibility Complex (MHC) of antigen-specific antigen presenting cells (APCs) with the T cell antigen receptor (TCR). The second one is an antigen-nonspecific costimulatory signal. Lack of such a costimulatory signal after TCR antigen recognition leads to partial or failed T-cell activation, which in turn causes T cell anergy that induces no response of T cells to a subsequent antigen attack any more. Such T cell anergy is most important for induction of antigen-specific tolerance to prevent transplantation rejection.

The most important costimulatory signal for T-cell activation is the binding between CD28 and CTLA4 of T cells and the B7 receptors (CD80 and CD86) present on the surface of APCs. CTLA4 has about a 20-fold higher affinity for the B7 receptor than CD28 (Linsley et al., J. Exp. Med. 174: 561, 1991; and Linsley et al., Immunity, 1:793, 1994). Unlike CD28, binding of CTLA4 to the B7 receptor results in delivery of a signal that inhibits or attenuates T-cell activation (Sebille et al., Philos. Trans. R. Soc. Lond., B Biol. Sci. 356:649, 2001).

In addition, Linsley et al. (J. Exp. Med., 174: 561, 1991) have reported preparation of a CTLA4Ig fusion protein in which an Fc portion of immunoglobulin G (IgG) was artificially fused to the C-terminus of CTLA4, in conjunction with immunosuppressive effects thereof. The immunoglobulin portion of such a CTLA4Ig fusion makes it possible to achieve effective purification on an affinity chromatography column, and production of a dimeric CTLA4 protein and a prolonged in vivo half-life. Blockage of the CD28/B7 costimulatory signal by CTLA4Ig resulted in prolonged graft survival in a variety of animal experimental models, including rat cardiac transplantation (Guillot et al., J. Immunol. 164:5228, 2000; Hayashi et al., Transpl. Int. 13 (Suppl. 1), S329, 2000; and Turka et al., Proc. Natl. Acad. Sci. U.S.A. 89:11102, 1992), mouse islet xenograft (Feng et al., Transplantation 67:1607, 1999; and Lenschow et al., Science, 257:789, 2000), rat renal transplantation (Tomasoni et al., J. Am. Soc. Nephrol. 11, 747, 2000) and monkey islet transplantation (Kirk et al., Proc. Natl. Acad. Sci. U.S.A. 94: 8789, 1997; and Levisetti et al., J. Immunol. 159: 5187, 1997), and thus suggesting a therapeutic potential important for practical clinical applications. In fact, clinical trials have shown highly promising results that CTLA4Ig is therapeutically effective (Abrams et al., J. Clin. Invest. 103: 1243, 1999, J. Exp. Med. 192: 681, 2000; and Guinan et al., N. Engl. J. Med., 340:1704, 1999). Conventional immunosuppressants, such as commonly and widely used steroid hormone drugs, e.g. cyclophosphamide, cyclosporin and FK506, exhibit adverse side effects due to their substantially non-specific suppression of the immune system. Whereas, CTLA4Ig specifically suppresses only the T-cell activation process and is therefore expected to exhibit superior immunosuppressive effects with relatively less adverse side effects.

However, a dose of such a CTLA4Ig fusion protein to a human is up to 10 mg/kg each time, which is relatively high as compared to other cytokine protein preparations (Abrams, J. R. et al., J. Clin. Invest., 103(9): 1243, 1999; Greene J. L. et al., Arthritis Rheum. 46: 1470, 2002; and Kremer, J. M. et al., N. Engl. J. Med., 349(20): 1907, 2003). For this reason, CTLA4Ig is extremely costly to produce using conventional animal cell culture techniques.

With a recent report, biologically active CTLA4Ig was produced from milk of transgenic animals in about 5-fold higher yield than animal cell systems (Lui V. C. H., et al., J. Immuno., Meth., 277:171, 2003).

With advanced plant biotechnology, a number of attempts have been actively made to produce high-value added beneficial proteins through large-scale cultivation of plant cells. Due to economical advantages associated with use of inexpensive medium components and easy and convenient production, isolation and purification of desired proteins, such plant cell culture-based production systems are receiving a great deal of attention as a substitute production system for pharmaceutical proteins such as cytokines, growth factors and immunomodulators that have been produced by use of conventional microbial or animal cell culture systems (Miele, L., Trends Biotechnol., 15: 45-50, 1997; and Doran, P. M., Curt Opin. Biotechnol., 11: 199-204, 2000). Further, production of recombinant proteins via plant cell culture involves, unlike prokaryotic cell systems such as E. coli, a post-translational modification process almost similar to that exhibited by animal cells. Thus, it is easy to maintain biological activities of the as-produced proteins and it is also advantageous in terms of safety, due to the decreased risk of incorporation of viruses or pathogenic bacteria possibly fatal to humans, as compared to the animal cell culture involving use of sera. In particular, post-translational modifications (PTMs) of the protein are features unique and intrinsic to eukaryotic cells, and therefore plant cells capable of performing such a protein post-translational modification mechanism are significantly advantageous in that they can substitute the recombinant protein production systems that are based on animal cell cultures.

It is known that protein glycosylation, among the post-translational modifications, is very important for physiological activity of glycoproteins and has significant effects on activity, conformational structures, stability, solubility and blood clearance rates of the proteins. The protein glycosylation may be broadly categorized into two types, N-linked glycosylation and O-linked glycosylation, depending on amino acid sequences of proteins with attachment of glycans. Particularly, N-linked glycosylation with attachment of a glycan to an amino acid asparagine (Asn) residue is more frequent in organisms and its effects on functions of the proteins have been extensively studied and understood.

Meanwhile, the N-linked glycosylation mechanism is reportedly substantially the same between plant and animal cells, but there is a slight difference in the structure of glycan moieties attached to the proteins (Kukuruzinska and Lennon, 1998; Lerouge et al., 1998; and Rayon et al., 1998). Among N-linked glycosylation glycan motifs, high-mannose-type N-glycans are found to have the same structure in both of plants and animals whereas complex-type N-glycans are known to have different conformational structures therebetween. Particularly, the glycan moiety of animal cell-derived glycoproteins has a structure where β1,4-galactose and sialic acid are further attached terminally to the Man₃GlcNAc₂ core structure, whereas the plant counterpart of the glycan moiety exhibits no attachment of such terminal residues and additionally contains β1,2-xylose and α-1,3-fucose residues which are not found in animals, that is plant-specific.

Due to such a difference in the N-linked glycosylation pattern between the plant and animal cells, a plant-derived recombinant protein exhibits a higher blood clearance rate as compared to the animal cell-derived counterpart, upon intravenous injection, thus resulting in deterioration of an in vivo half life, and is likely to cause immunogenicity due to the presence of glycan residues which are not found in animal cells.

Some of attempts have been recently made on modification of a plant glycosylation pattern to resemble that of a human, through expression of animal-derived N-linked glycosylation enzymes in transformed plant cells. However, there is little research and study on glycan modification of recombinant proteins with limitation to some plants including tobacco (Nicotiana tabaccum) and thale cress (Arabidopsis thaliana).

### DISCLOSURE OF THE INVENTION

### Technical Problem

As a result of extensive and intensive research and study to solve conventional problems, the present inventors have developed vectors and plant cells having a potent promoter system capable of inducing high expression of CTLA4Ig, in conjunction with construction of a vector that is capable of expressing a β1,4-galactosyltransferase (hGalT) enzyme which is one of human-derived N-linked glycosylation enzymes, in a plant cell expression system, and plant cells containing the same, and have discovered that plant cells transformed with the thus-constructed vectors are capable of expressing plant-derived recombinant human CTLA4Ig (CTLA4Ig^{P}) at a high concentration, and an in vivo half life of the plant-derived CTLA4Ig can be increased by rendering the cells to have the same glycan structure as that of the animal cell-derived protein. The present invention has been completed based on these findings.

Therefore, an object of the present invention is to provide a recombinant vector pBI-3D-hGalT or pBI-35S-hGalT containing a human β1,4-galactosyltransferase gene; a cell line transformed with a recombinant vector pMYN414 containing a cytotoxic T-lymphocyte antigen 4-immunoglobulin (CTLA4Ig) fusion protein gene and the recombinant vector pBI-3D-hGalT or pBI-35S-hGalT; and a method for producing a plant-derived recombinant human CTLA4Ig (CTLA4Ig^{P}) fusion protein with a human glycan structure using the same.

### Technical Solution

In accordance with an aspect of the present invention, the above and other objects can be accomplished by the provision of a recombinant vector pBI-3D-hGalT containing a human β1,4-galactosyltransferase (hGalT) gene and having a cleavage map as shown in FIG. 1 and a recombinant vector pBI-35S-hGalT containing a human β1,4-galactosyltransferase (hGalT) gene and having a cleavage map as shown in FIG. 2.

In accordance with another aspect of the present invention, there is provided a plant cell transformed with a recombinant vector pMYN414 containing a human cytotoxic T lymphocyte antigen 4-immunoglobulin (CTLA4Ig) fusion protein gene and having a cleavage map as shown in FIG. 3 and the recombinant vector pBI-3D-hGalT, and a plant cell transformed with the recombinant vector pMYN414 and the recombinant vector pBI-35S-hGalT.

In accordance with a further aspect of the present invention, there is provided a method for producing a plant-derived recombinant human CTLA4Ig (CTLA4Ig^{P}) fusion protein comprising suspension-culturing the above-mentioned transformed plant cells and separating a CTLA4Ig^{P} fusion protein from the cell culture, a CTLA4Ig^{P} fusion protein produced by the aforesaid method, and an immunosuppressive pharmaceutical composition comprising the CTLA4Ig^{P} fusion protein.

In accordance with a further aspect of the present invention, there is provided a use of the thus-produced CTLA4Ig^{P} fusion protein produced by the method for producing a CTLA4Ig^{P} fusion protein according to the present invention, for the preparation of an immunosuppressant.

In accordance with yet another aspect of the present invention, there is provided a method for inhibiting an immune response, comprising administering to a mammal an effective amount of the thus-produced CTLA4Ig^{P} fusion protein produced by the method for producing a CTLA4Ig^{P} fusion protein according to the present invention.

The present invention will be described in more detail.

The present invention provides a recombinant vector pBI-3D-hGalT containing a human β1,4-galactosyltransferase (hGalT) gene and having a cleavage map as shown in FIG. 1.

The recombinant vector pBI-3D-hGalT can be expressed under the control of a rice amylase 3D (RAmy3D) promoter.

In one embodiment of the present invention, the hGalT gene can have a nucleotide sequence as set forth in SEQ ID NO: 1.

Further, the present invention provides a recombinant vector pBI-35S-hGalT containing a human β1,4-galactosyltransferase (hGalT) gene and having a cleavage map as shown in FIG. 2.

The recombinant vector pBI-35S-hGalT can be expressed under the control of a CaMV 35S promoter.

In one embodiment of the present invention, the hGalT gene can have a nucleotide sequence as set forth in SEQ ID NO: 1.

These recombinant vectors pBI-3D-hGalT and pBI-35S-hGalT are constructed such that resistance against an antibiotic G418 is adopted as a selection marker of a transformed plant cell line, and a human glycan residue β1,4-glactose can be added to the terminal of a glycan structure during N-linked glycosylation of the plant-derived recombinant protein.

Further, the present invention provides a plant cell transformed with a recombinant vector pMYN414 containing a human cytotoxic T lymphocyte antigen 4-immunoglobulin (CTLA4Ig) fusion protein gene and having a cleavage map as shown in FIG. 3 and the aforesaid recombinant vector pBI-3D-hGalT. Further, the present invention provides a plant cell transformed with the recombinant vector pMYN414 and the recombinant vector pBI-35S-hGalT.

In one embodiment of the present invention, the CTLA4Ig fusion protein gene can have a nucleotide sequence as set forth in SEQ ID NO: 2.

In the nucleotide sequence of SEQ ID NO: 2, bases 1 to 93 correspond to a signal peptide, bases 94 to 465 correspond to a CTLA4 extracellular domain, and bases 466 to 1167 correspond to an IgGl Fc fragment.

In addition, the recombinant vector pMYN414 is constructed to use an antibiotic hygromycin resistance as a selection marker of a transformed plant cell line and to allow extracellular secretion of the recombinant CTLA4Ig fusion protein outside plant cells by the RAmy1A signal peptide, upon suspension culturing.

As a result, the transformed plant cell lines express and secrete the CTLA4Ig^{P} (plant-derived recombinant human CTLA4Ig) into the culture medium upon suspension culturing of cells, and the as-expressed CTLA4Ig^{P} has a terminal β1,4-galactose residue which is a human glycan residue.

There is no particular limit to the plant cell that can be transformed with the aforesaid recombinant vector. Examples of utilizable plant cells may include rice (Oryza sativa L.), tobacco (Nicotiana tabacum), maize (Zea mays), soybean (Glycine max), wheat (Triticum aestivum), tomato (Lycopersicon esculentum), rape (Brassica napus) and potato (Solanum tuberosum).

In one embodiment of the present invention, the plant cell to be transformed with the aforesaid recombinant vector is rice (Oryza sativa L.).

In one embodiment of the present invention, the transformed plant cell is a rice cell line Oryza sativa L. under Accession Number KCTC 11141 BP.

In another embodiment of the present invention, the transformed plant cell is a rice cell line Oryza sativa L. under Accession Number KCTC 11142BP.

Further, the present invention provides a method for producing a plant-derived recombinant human CTLA4Ig (CTLA4Ig^{P}) comprising suspension-culturing the above-mentioned transformed plant cells and separating CTLA4Ig^{P} from the culture medium.

The suspension culture of transformed plant cells may be carried out by any conventional plant cell cultivation method known in the art.

In one embodiment of the present invention, the suspension culturing may be carried out in a medium containing, for example, sugars, growth regulators, and antibiotics for selection.

The suspension culture may employ a basal medium widely used in the plant cell cultivation, such as Chu N6 medium, AA (amino acid) medium, MS (Murashige and Skoog) medium, SH (Schenk and Hildebrandt) medium, LS (Linsmaier and Skoog) medium, B5 medium, White's medium, or the like.

Examples of sugar supplemented to the culture medium may include sucrose, glucose, fructose, maltose, lactose, galactose, mannose, starch, glycerol, sorbitol, mannitol, pyruvic acid, and the like.

Examples of the growth regulator that can be used in the present invention may include 2,4-dichlorophenoxyacetic acid (2,4-D), kinetin, indoleacetic acid (IAA), naphthaleneacetic acid (NAA), indole butyric acid (IBA), zeatin, 6-benzyl amino purine (BAP), gibberellic acid (GA3), abscisic acid (ABA), and the like.

Examples of the antibiotic for selection of transformants may include hygromycin, G-418, kanamycin, zeocine, and the like.

In one embodiment of the present invention, the culture method includes co-transfection of the recombinant vector pMYN14 and pBI-3D-hGalT or pBI-35S-hGalT into target plant cells by Agrobacterium-mediated transformation, and selection of antibiotic-resistant transformed plant cell lines through cell culture in the selective medium. Mass production of the plant-derived recombinant human CTLA4Ig (CTLA4Ig^{P}) is then conducted by suspension culture of the thus-selected transformed plant cells. Preferably, the suspension culture is carried out using, as a basal medium, a Chu N6 or AA medium, or an MS (Murashige and Skoog) medium widely used in plant cell cultivation, containing 10 to 60 g/L of sucrose as a carbon source, 0.1 to 2.0 mg/L of 2,4-D, 0.01 to 2.0 mg/L of kinetin and 0.01 to 2.0 mg/L of gibberellic acid as growth regulators, and 50 mg/L of hygromycin and 50 mg/L of G418 as selectable markers, at a temperature of 22 to 28° C. and at 80 to 150 rpm under dark conditions.

For example, when it is desired to carry out suspension culture of Oryza sativa BR-Os/3D-hGalT (KCTC 11141BP) or Oryza sativa BR-Os/35S-hGalT (KCTC 11142BP), this cell line is cultured at 28° C. and 120 rpm under dark conditions, using an N6 liquid medium or AA rice suspension culture medium containing 30 g/L of sucrose, 2 mg/L of 2,4-D, 0.2 mg/L of kinetin, 0.1 mg/L of gibberellic acid, 50 mg/L of hygromycin and 50 mg/L of G418. On Day 7 of culture, the culture medium is replaced with a sucrose-free N6 or AA medium to thereby induce expression of the recombinant protein, preferably resulting in expression of CTLA4Ig^{P}.

When the Oryza sativa BR-Os/3D-hGalT or Oryza sativa BR-Os/35S-hGalT culture is centrifuged to separate the culture supernatant which is then purified by protein A affinity chromatography, followed by freeze-drying to obtain the desired protein CTLA4Ig^{P}, the Oryza sativa BR-Os/3D-hGalT or Oryza sativa BR-Os/35S-hGalT in accordance with the present invention yields about 10 mg of CTLA4Ig^{P} per liter of the culture.

Further, the present invention provides a plant-derived recombinant human CTLA4Ig (CTLA4Ig^{P}) fusion protein isolated and produced from the culture medium where the transformed plant cells are suspension-cultured.

According to the lectin blot analysis using lectin which exhibits specific reactivity for glycan moieties, it was confirmed that the CTLA4Ig^{P} fusion protein produced according to the present invention has a human-like glycan structure, i.e. terminal β1,4-glactose residue, as shown in the recombinant CTLA4Ig^{M} produced by a conventional CHO cell expression system.

In addition, it was confirmed that the CTLA4Ig^{P} fusion protein having a human glycan structure, produced in the plant cell system according to the present invention, exhibits significant improvement in an in vivo half life as compared to conventional plant-derived CTLA4Ig, when the CTLA4Ig^{P} fusion protein was intravenously injected to rats, followed by determination of the plasma CTLA4Ig^{P} level.

Consequently, the CTLA4Ig^{P} fusion protein of the present invention can be produced on a large scale at lower production costs using the plant expression system and has a human glycan structure which provides immunosuppressive activity comparable to that of the CTLA4Ig^{M} fusion protein expressed through conventional animal cell expression systems.

Further, the present invention provides an immunosuppressive pharmaceutical composition comprising a CTLA4Ig^{P} fusion protein as an active ingredient.

The pharmaceutical composition of the present invention may further comprise one or more pharmaceutically acceptable carriers besides the aforesaid fusion protein, to be formulated into a variety of dosage forms for desired applications.

The pharmaceutical composition may be administered via a conventional route, for example intravenously, intraarterially, percutaneously, intradermally, subcutaneously, intramuscularly, intraperitoneally, intrathoracically, intranasally, locally, rectally, orally, intraocularly, or by inhalation.

When the composition of the present invention is formulated into an injectable preparation, buffer for injection and other additive components may be added which are well-known in the art. The injectable preparation of the present composition may further comprise additive components such as solubilizers, pH-adjusting agents, suspending agents, etc., besides the buffer for injection. As the buffer for injection, physiological saline may be used.

Dosage forms of the composition of the present invention may include granules, powders, coated tablets, tablets, capsules, suppositories, syrups, juice, suspensions, emulsions, and sustained-release formulations of an active compound.

For formulation of the composition into a tablet or capsule, the active ingredient may be combined with any oral, non-toxic and pharmaceutically acceptable inert carrier such as ethanol, glycerol, water, etc. If desired or necessary, suitable binders, lubricants, disintegrants and colorants may be added. Examples of the suitable binder may include, but are not limited to, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Examples of the disintegrant may include, but are not limited to, starch, methyl cellulose, agar, bentonite, and xanthan gum.

For formulation of the composition into a liquid preparation, a pharmaceutically acceptable carrier which is sterile and biocompatible may be used such as saline, sterile water, Ringer's solution, buffered physiological saline, albumin infusion solution, dextrose solution, maltodextrin solution, glycerol, and ethanol. These materials may be used alone or in any combination thereof. If necessary, other conventional additives may be added such as antioxidants, buffers, bacteriostatic agents, and the like. Further, diluents, dispersants, surfactants, binders and lubricants may be additionally added to the composition to prepare injectable formulations such as aqueous solutions, suspensions, and emulsions, or oral formulations such as pills, capsules, granules, and tablets. Furthermore, the composition may be preferably formulated into a desired dosage form, depending upon diseases to be treated and ingredients, using any appropriate method known in the art, as disclosed in Remington's Pharmaceutical Sciences, Mack Publishing Co., Easton, Pa.

The pharmaceutical composition of the present invention can be used for immunosuppression, including prevention or treatment of autoimmune diseases (such as rheumatoid arthritis, psoriasis, lupus, asthma, and the like) or transplantation rejection.

Accordingly, the present invention further provides a use of a pharmaceutical composition comprising a CTLA4Ig^{P} fusion protein as an active ingredient, which is intended for the preparation of an immunosuppressant. Therefore, the aforesaid pharmaceutical composition comprising the fusion protein of the present invention may be used for the preparation of such an immunosuppressant.

Further, the present invention provides a use of a CTLA4Ig^{P} fusion protein for the preparation of an immunosuppressant.

Further, the present invention provides a method for inhibiting an immune response, comprising administering to a mammal a therapeutically effective amount of a CTLA4Ig^{P} fusion protein.

As used herein, the term “mammal” refers to a subject that is in need of treatment, examination or experiment, preferably human.

As used herein, the term “therapeutically effective amount” refers to an amount of an active ingredient or pharmaceutical composition that will elicit the biological or medical response of a tissue system, animal or human that is being sought by a researcher, veterinarian, medical practitioner or clinician, and encompasses an amount of the active ingredient or pharmaceutical composition which will ameliorate the symptoms of the disease or disorder being treated. As will be apparent to those skilled in the art, the therapeutically effective dose and administration times of the active ingredient in accordance with the present invention may vary depending upon desired therapeutic effects. Therefore, an optimal dose of the active drug to be administered can be easily determined by those skilled in the art. For example, an effective dose of the drug is determined taking into consideration various factors such as kinds of disease, severity of disease, contents of active ingredients and other components contained in the composition, kinds of formulations, age, weight, general health status, sex and dietary habits of patients, administration times and routes, release rates of the composition, treatment duration, and co-administered drugs. For adults, the CTLA4Ig^{P} fusion protein may be preferably administered at a dose of 1 mg/kg to 50 mg/kg once or several times a day.

In addition, the pharmaceutical composition of the present invention may be administered in combination with known immunosuppressant(s).

### ADVANTAGEOUS EFFECTS

As described hereinbefore, a plant cell-derived recombinant human CTLA4Ig fusion protein (CTLA4Ig^{P}), which has a human glycan structure and is produced according to the present invention, can exhibit an improved in vivo half life as compared to conventional plant-derived proteins, due to the presence of a human-like glycan structure. Consequently, the present invention using the plant expression system enables low-cost mass production of a CTLA4Ig^{P} fusion protein having an immunosuppressive activity comparable to that of a CTLA4Ig^{M} fusion protein expressed in conventional animal cell expression systems.

### DESCRIPTION OF THE DRAWINGS

FIG. 1 is a cleavage map of a pBI-3D-hGalT vector in accordance with the present invention;

FIG. 2 is a cleavage map of a pBI-35S-hGalT vector in accordance with the present invention;

FIG. 3 is a cleavage map of a pMYN414 vector in accordance with the present invention;

FIG. 4 is a photograph showing PCR amplification results of CTLA4Ig and hGalT genes incorporated into a transformed rice cell line;

FIG. 5 is a graph showing the quantification analysis results of a CTLA4Ig^{P} fusion protein expressed in suspension culture of a transformed rice cell line in accordance with the present invention;

FIG. 6 is a photograph showing RT-PCR confirmation of mRNAs for CTLA4Ig^{P} and hGalT (A) and a photograph showing Western blot patterns of a CTLA4Ig^{P} fusion protein (B), after suspension culture of a transformed rice cell line in accordance with the present invention;

FIG. 7 is a photograph showing Western and Lectin blot patterns for a human glycan structure of a CTLA4Ig^{P} fusion protein in accordance with the present invention;

FIG. 8 is a graph showing plasma level-time profiles and an in vivo half life of a CTLA4Ig^{P} fusion protein in accordance with the present invention, in animal experiments using rats;

FIG. 9 is a graph showing the results for an in vitro immunosuppressive activity test using mouse splenocytes, which is intended to confirm antiproliferative effects of a CTLA4Ig^{P} fusion protein of the present invention on T cells; and

FIG. 10 is a graph confirming that a CTLA4Ig^{P} fusion protein of the present invention inhibits secretion of T cell-derived immunocytokines, mainly IL-2 (A) and IFN-γ (B).

### MODE FOR INVENTION

Now, preferred embodiments of the present invention will be described in more detail, such that those skilled in the art can easily practice the present invention. These and other objects, advantages and features of the present invention will become apparent from the detailed embodiments given below which are made in conjunction with the following Examples. The present invention may be embodied in different forms and should not be misconstrued as being limited to the embodiments set forth herein, and those skilled in the art will appreciate that various modifications, additions and substitutions are possible without departing from the scope and spirit of the invention as disclosed in the accompanying claims. Therefore, it should be understood that the embodiments disclosed herein are provided only for illustrating the present invention and should not be construed as limiting the scope and spirit of the present invention.

### EXAMPLE

### Production of a CTLA4Ig^{P} Fusion Protein Having a Human Glycan Structure in Transformed Plant Cells

### Example 1

### Construction of Recombinant Vectors pBI-3D-hGalT and pBI-35S-hGalT Containing a β1,4-Galactosyltransferase (hGalT) Gene

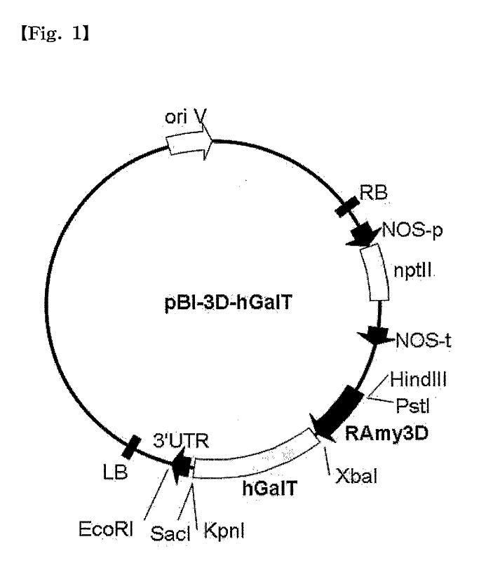

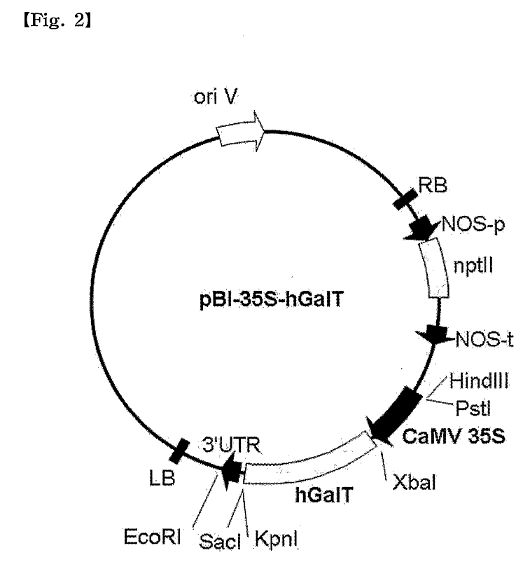

Cleavage maps of recombinant vectors pBI-3D-hGalT and pBI-35S-hGalT containing a CTLA4Ig gene, as constructed in this Example, are schematically shown in FIG. 1 and FIG. 2, respectively.

Construction of the recombinant vectors pBI-3D-hGalT and pBI-35S-hGalT is as follows.

Cloning of a β1,4-galactosyltransferase (hGalT) gene for glycosylation modification of CTLA4Ig was amplified by construction of two primer sets based on the sequence data available from the NCBI Gene bank.

A gene galF (1-381 bp) at the N-terminal region of β1,4-galactosyltransferase was amplified using human genomic DNA as a template, whereas a gene galR (382-1193 bp) of the C-terminal region was amplified by culturing the human fibroblast cell line MRC5, extracting mRNA from the cultured cells, and synthesizing cDNA from the extracted mRNA using reverse transcriptase, followed by amplification using the resulting cDNA as a template.

For ligation of two amplified genes, a restriction endonuclease recognition site SpeI was introduced into the 3′ portion of galF and 5′ portion of galR.

Using a forward primer GalS1 (aaatctagagcgatgccaggcgcgtccct) containing a XbaI site and a reverse primer GalS2 (aatactagtageggggactcctcagggca) containing a SpeI site in order to amplify the N-terminus of β1,4-galactosyltransferase, PCR amplification of a template gene was carried out as follows: 30 cycles of denaturation at 94° C. for 45 seconds, annealing at 55° C. for 45 seconds and elongation at 72° C. for 45 seconds. PCR amplification of the C-terminus of β1,4-galactosyltransferase was also carried out under the same conditions, using a forward primer Gal1S (aagactagtgggccccatgctgattga) containing a SpeI site and a reverse primer Gal2K (gtaggtaccgtgtaccaaaacgctagct) containing a KpnI site.

Each of partial genes identified by agarose gel electrophoresis was ligated into a pGEM-T Easy vector (Promega, USA) which was then transformed into E. coli DH5a cells, followed by blue/white colony selection on a LB agar plate containing Amp/X-Gal to thereby obtain plasmid pGEMT-galF and pGEMT-galR clones.

For ligation of each gene, pGEMT-galF plasmid and pGEMT-galR plasmid were subjected to restriction cleavage with endonuclease SpeI at 37° C. for 4 hours. A galR gene fragment was ligated into the Spa site of pGEMT-galF, thereby obtaining a pGEMT-hGalT plasmid harboring an intact β1,4-galactosyltransferase gene (SEQ ID NO: 1).

For expression of the β1,4-galactosyltransferase gene in rice, first a gene of interest was subcloned into an expression vector pMYN75 containing a RAmy3D promoter, a 3′-untranslated region (UTR) and a hygromycin selectable marker, and the desired gene from the thus-constructed pMY-hGalT was subcloned again into an expression vector pBI121 containing a CaMV35S promoter, a 3′-UTR and a G418 selectable marker.

First, the pGEMT-hGalT plasmid and the pMYN75 plasmid were subjected to restriction cleavage with endonucleases XbaI and KpnI at 37° C. for 4 hours. After electrophoresis on a 1.0% agarose gel, a hGalT DNA fragment of pGEMT-hGalT was purified using Promega wizard SV Gel and PCR Clean up system and ligated into the endonuclease-restricted pMYN75 vector, followed by selection of a pMY-hGalT clone harboring a β1,4-galactosyltransferase gene. The restriction endonuclease mapping revealed that the plasmid pMY-hGalT has a desired β1,4-galactosyltransferase gene inserted between the RAmy3D promoter and 3′-UTR.

Thereafter, for cloning of a β1,4-galactosyltransferase gene into a pBI121 vector containing a CaMV35S promoter and a G418 selectable marker, pMY-hGalT and pBI121 were restricted with endonucleases XbaI and EcoRI at 37° C. for 4 hours, and DNA of the hGalT region of pMY-hGalT was ligated into the pBI121 vector, followed by selection of pBI-35S-hGalT clones (about 14100 bp) through E. coli transformation.

Next, for construction of expression vector containing a RAmy3D promoter and a G418 selectable marker, pMY-hGalT and pBI121 were restricted with endonuclease HindIII and EcoRI at 37° C. for 4 hours, and the 3D promoter and hGalT DNA fragment of pMYN-hGalT were ligated into the endonuclease-restricted pBI121 vector, followed by selection of pBI-3D-hGalT clones (about 14308 bp) through E. coli transformation.

### Example 2

### Construction of a Recombinant Vector pMYN414 Harboring a CTLA4Ig Gene

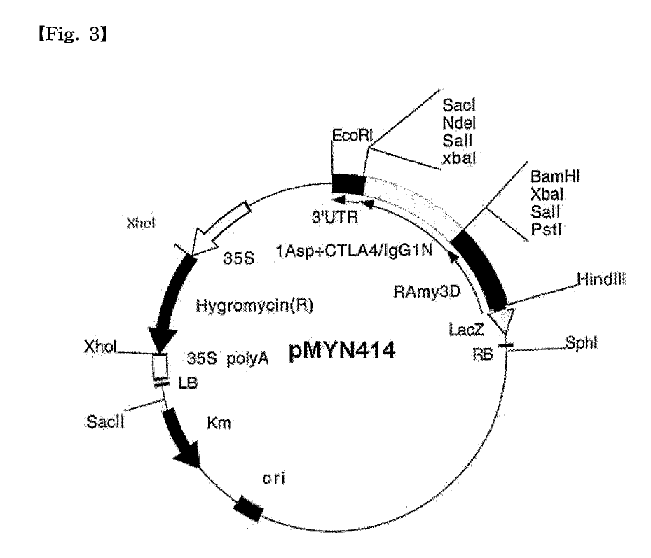

A gene cleavage map of the recombinant vector pMYN414 harboring a CTLA4Ig gene constructed in this Example is schematically shown in FIG. 3.

Construction of the recombinant expression vector pMYN414 is as follows.

In order to induce secretion of the CTLA4Ig fusion protein into the culture medium, a synthetic fusion gene was constructed by ligation of a rice amylase signal peptide (hereinafter, referred to as “Ramy1A signal peptide”) into the N-terminus of hCTLA4 through the overlapping polymerase chain reaction (PCR).

First, cDNA was synthesized from mRNA of CTLA-4 extracted from mononucleocytes isolated from the adult male blood, and PCR was carried out using the obtained cDNA as a template. Using a forward primer CTLA4-F1 (5′-TCCAACTTGACAGCCGGGGCAA TGCACGTGGCCCAGCCTGC-3′), concomitantly containing a C-terminal sequence of RAmy1A signal peptide and an N-terminal sequence of CTLA-4, and a reverse primer CTLA4-R1 (5′-CTCTGCAGAATCTGGG CACGGTTCTG-3′) recognizing the C-terminus of CTLA-4, PCR was carried out as follows: one cycle of pre-denaturation at 94° C. for 2 min; 30 cycles of denaturation at 94° C. for 45 seconds, annealing at 55° C. for 45 seconds and elongation at 72° C. for 45 seconds; and one cycle of final elongation at 72° C. for 5 min.

Using the thus-obtained PCR products, and a RAmy1A secretion signal gene obtained from rice cDNA as a template, overlapping PCR was carried out to construct a synthetic fusion gene in which a high-secretion leader sequence of the rice amylase was bound to the hCTLA4 gene and the thus-prepared fusion gene was inserted into a pGEM-T Easy vector (Promega, USA) to construct a vector pMYN407.

A pMYN406 vector harboring a human immunoglobulin IgGl Fc gene was cleaved with endonuclease PstI/SalI and the resulting IgG1 Fc gene was inserted into the pMYN407 vector which had been cleaved with the same endonuclease, thereby constructing a vector pMYN408.

Utilizing a Transformer™ Site-Directed Mutagenesis kit (BD Biosciences, USA), a vector pMYN411 in which the amino acid ²⁷²Pro of the IgGl Fc region in the CTLA4Ig fusion protein gene had been substituted with ²⁷² Ser was constructed from the pMYN408 vector.

Finally, the CTLA4Ig fusion protein gene (SEQ ID NO: 2) ligated to the RAmy1A signal sequence on the pMYN411 vector was cleaved with BamHI/SacI and the BamHI/SacI fragment was inserted into a pMYN75 expression vector containing the same restriction endonuclease cleavage sites to thereby construct a pMYN414 vector.

Insertion of the CTLA4Ig gene (about 11519 bp) between the RAmy3D promoter and the 3′-UTR region was confirmed by restriction endonuclease mapping.

### Example 3

### Construction of a Transformed Plant Cell Line Producing a CTLA4Ig^{P} Fusion Protein Having a Human Glycan Structure

In order to obtain a transformed plant cell line producing a CTLA4Ig^{P} fusion protein having a human glycan structure, a recombinant vector pMYN414 expressing CTLA4Ig and a recombinant vector pBI-3D-hGalT or pBI-35S-hGalT expressing β1,4-galactosyltransferase were transformed into rice cells using agrobacterium-mediated transformation.

Firstly, rice seeds were dehulled, surface-sterilized with 70% ethanol and NaOCl solution, and washed with sterile distilled water. The thus-treated rice seeds were placed on a callus-induction N6CI agar medium. After incubation at 27° C. under a 16/8-hr light/dark cycle for 14 days, scutellum-derived calli were isolated from germinated seeds and transferred to a fresh N6CI agar medium, followed by incubation for 7 days. Only the calli were transferred to a metal mesh which was then ready for agrobacterium infection.

Agrobacterium transformants into which the plant expression vector was transformed were suspension-cultured in a LB medium supplemented with rifampicin and kanamycin at 27° C. and at 150 rpm for 2 days, smeared on an ABKR agar medium and cultured again for 2 days. Agrobacterium transformants cultured on the ABKR agar medium were scratched with a spatula and transferred and homogeneously distributed to O.D. of 1.5 to 2.0 in an AAM medium. Then, the resulting Agrobacterium suspension was infused into a 100Φ petri dish.

The calli prepared on the metal mesh were soaked in an agrobacterium suspension for 15 min and shaken every 2 minutes. Infection-completed calli were transferred to a callus-agrobacterium co-culture medium N6CO, and incubated in the dark at 27° C. for 3 days.

For selection of the transformed calli, the infected calli were pooled in a 50-mL tube and washed twice with sterile purified water. Cefotaxime was added to the calli which were then washed once, transferred to an N6SE selective medium supplemented with G418, hygromycin and cefotaxime, incubated in the dark at 27° C. under a 16/8-hr light/dark cycle for 4-6 weeks and then observed until G418-hygromycin resistant calli grew up to a diameter of 1 cm.

### Example 4

### Confirmation of Gene Introduction by Genomic DNA PCR

In order to confirm whether CTLA4Ig and hGalT genes were successfully inserted into the rice seed-derived calli of Example 3, genomic DNA was extracted from the rice calli, followed by PCR amplification.

For extraction of the genomic DNA, rice callus tissues were frozen in liquid nitrogen, and ground. The rice genomic DNA was obtained using a DNeasy Plant Mini Kit (Qiagen Inc., USA).

Using the same forward and reverse primers as in cloning of the CTLA4Ig and hGalT genes and the rice callus-derived genomic DNA as a template, the genomic DNA PCR was carried out as follows: one cycle of initial denaturation at 94° C. for 2 min; 25 cycles of denaturation at 94° C. for 45 seconds, annealing at 55° C. for 45 seconds and elongation at 72° C. for 45 seconds; and one cycle of final elongation at 72° C. for 5 min. The resulting PCR products were stained with EtBr after electrophoresis on a 1.0% agarose gel.

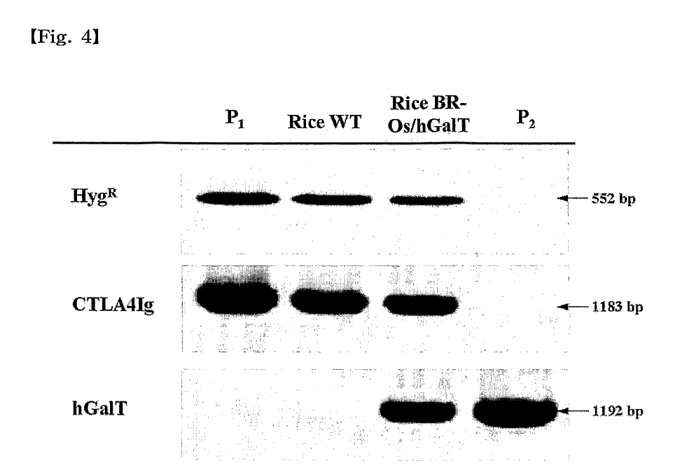

FIG. 4 is a photograph of PCR-amplified CTLA4Ig and hGalT genes incorporated into the transformed rice callus cell lines. P1: PCR positive control of a pMYN414 vector for CTLA4Ig, P2: PCR positive control of a pBI-3D-hGalT vector for hGalT, Rice WT: PCR negative control of genomic DNA of natural rice cell without expression of hGalT, and Rice BR-Os/hGalT: PCR results of transformed rice callus cell lines of Example 3.

As can be seen from FIG. 4, only the transformed rice callus cell line of the present invention simultaneously contained both the CTLA4Ig gene and the hGalT gene, and the PCR products exhibited a size of about 1183 by and 1192 bp, thereby confirming consistency with the expected size, similar to positive controls. Therefore, it was determined that CTLA4Ig and hGalT genes were successfully inserted into chromosomes of the transformed rice callus cell lines of Example 3. Among the transformed calli, the transformed calli with the highest gene expression rate were designated as Oryza sativa BR-Os/3D-hGalT and Oryza sativa BR-Os/35S-hGalT, respectively. Two transformants Oryza sativa BR-Os/3D-hGalT and BR-Os/35S-hGalT were each deposited with the Korean Collection for Type Cultures (KCTC), the Korean Research Institute of Bioscience and Biotechnology (KRIBB, Korea) under Accession Nos. KCTC 11141BP and KCTC 11142BP (deposited on Jun. 20, 2007).

### Example 5

### Suspension Culture of Transformed Rice Cell Line and Production of CTLA4Ig^{P} Fusion Protein Having a Human Glycan Structure

The transformed rice cell line obtained from Example 3 was suspension cultured to induce expression of a CTLA4Ig^{P} fusion protein having a human glycan structure. The selected callus cell line was transferred to a Chu (N6) liquid medium, followed by induction of suspension culture. Callus tissues were inoculated into an N6 medium containing 30 g/L of sucrose as a carbon source, 2 mg/L of 2,4-D and 0.2 mg/L of kinetin as growth regulators, 50 mg/L of hygromycin B and 50 mg/L of G418 as selectable markers, and cultured in a shaking incubator at 28° C. and 110 rpm. 2 week-interval subculturing was carried out by exchange of the culture medium with a fresh one.

In order to select a high-expression cell line from the suspension cells cultured in the N6 medium, cells were transferred to an AA medium to thereby induce fine suspension culture. The rice suspension cells, which were 2-3 month old with induction of the suspension culture, were transferred and subcultured in an AA medium containing 30 g/L of sucrose, 2.0 mg/L of 2,4-D, 0.2 mg/L of kinetin, 0.1 mg/L of gibberellic acid, 50 mg/L of hygromycin B, and 50 mg/L of G418. Upon subculturing, only the fine suspension cells separated from cell aggregates were selected and allowed to induce a high-expression suspension cell line.

In order to confirm secretion of the CTLA4Ig^{P} fusion protein into the culture medium, the culture medium was replaced with a sucrose-free medium to thereby induce expression of the desired protein after 10 days of culture. 1 to 9 days after medium replacement, the culture medium was harvested and subjected to ELISA so as to quantitatively confirm the amount of the CTLA4Ig^{P} fusion protein secreted into the medium. Molecular weight of the purified protein was determined by Western blotting. Further, mRNA was extracted from the rice cells and RT-PCR was carried out to confirm the expression of the CTLA4Ig and hGalT genes.

For ELISA, goat anti-human IgG (available from KPL) was diluted to 1:1000 in a coating buffer and 100 μl/well was aliquoted into a 96-well plate. After standing overnight at 4° C., the plate wells were washed three times with a washing buffer PBST (0.05% Tween 20-containing PBS). 200 μl of an assay diluent (PBS buffer containing 2% FBS) was aliquoted into each well of the 96-well plate, reacted at room temperature for 1 hour and then washed three times with the washing buffer PBST. 100 μl/well of culture samples was aliquoted, 2-fold serially diluted with the assay diluent and reacted at 37° C. for 1 hour. The plate was washed again with the washing buffer three times, and 100 μl/well of peroxidase-labeled goat anti-human IgG (available from KPL) diluted to 1:1000 in the assay diluent was added thereto, followed by reaction at 37° C. for 1 hour. This was followed by washing with the washing buffer three times, reaction with 100 μl of a substrate for 15 min and measurement of absorbance at 405 nm.

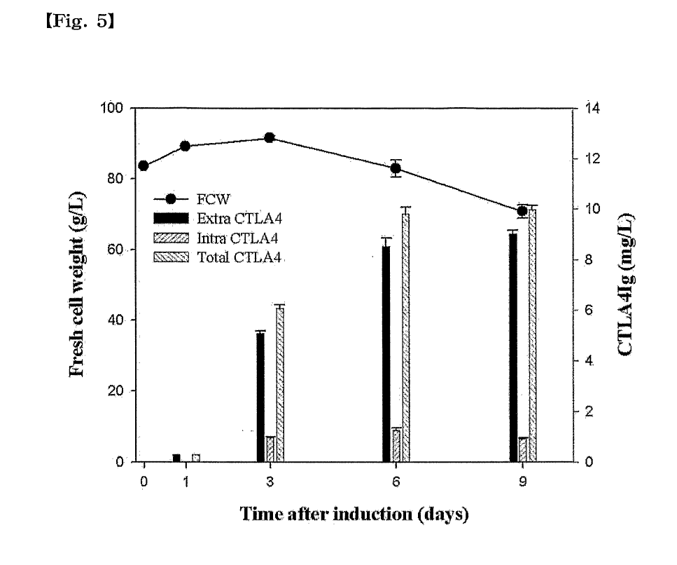

FIG. 5 graphically shows cell growth (line graph) and intracellular and extracellular production of the CTLA4Ig^{P} fusion protein (bar graph), when the transformed rice suspension cell lines were suspension-cultured in an AA medium. When the culture medium was replaced with a sucrose-free medium to induce expression of the recombinant protein on Day 7 of culture, production of the CTLA4Ig^{P} fusion protein in the medium rapidly increased 3 to 9 days later, yielding up to about 10 mg/L.

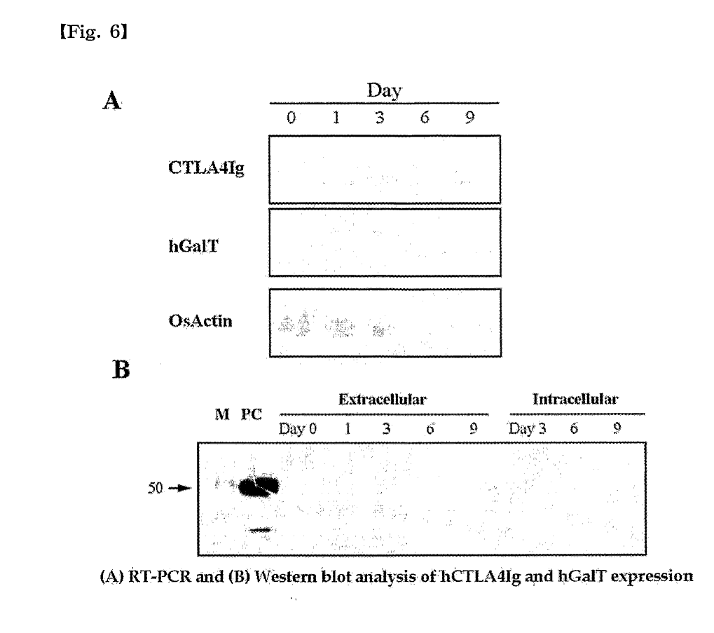

FIG. 6 is a photograph showing confirmation of RT-PCR (A) and Western blotting (B) for expression of mRNAs for CTLA4Ig^{P} and hGalT, upon suspension culture of the transformed rice suspension cell line. FIG. 6A shows RT-PCR confirmation for the expression of CTLA4Ig^{P} and hGalT genes following extraction of mRNA from the cells with induction of the recombinant protein expression by replacement of the culture medium with a sucrose-free medium. The mRNA expression rapidly increased between 1 to 3 days and then gradually decreased 6 days later. This is consistent with the quantitative analysis results of FIG. 5. Therefore, these results represent that the transformed cells of the present invention exhibit normal expression of the CTLA4Ig fusion protein and hGalT enzyme. Similar to FIG. 6A, FIG. 6B also shows Western blot confirmation for the extracellular production of CTLA4Ig^{P} upon suspension culture of the transformed rice suspension cell line. M: Molecular weight size marker and PC: CHO cell-derived CTLA4Ig^{M} positive control. As can be seen from the results of FIG. 5 and FIG. 6A, it was confirmed that the intracellular and extracellular expression of CTLA4Ig^{P} increases between 3 to 9 days, upon induction of the protein expression. A modified molecular weight of the transformed rice cell-derived CTLA4Ig^{P} fusion protein in accordance with the present invention was confirmed to be about 50 kDa, which is identical with that of the positive control.

From the results of ELISA and RT-PCR and Western blotting for CTLA4Ig and hGalT, it was confirmed that the transformed rice cell line of the present invention effectively expresses CTLA4Ig and hGalT and produces a CTLA4Ig^{P} fusion protein having the same molecular weight as that of the animal cell-derived CTLA4Ig^{M}.

### Experimental Example

### Assay for Glycan Structure and Half Life of CTLA4Ig Fusion Protein

### Experimental Example 1

### Glycan Analysis of CTLA4Ig^{P} Fusion Protein by Western Blotting and Lectin Blotting

After suspension culture of Oryza sativa BR-Os/3D-hGalT or BR-Os/35S-hGalT, the CTLA4Ig^{P} fusion protein was recovered from the culture medium and purified using a protein A column.

Purity and molecular weight of the purified CTLA4Ig^{P} fusion protein were determined by 10% SDS-PAGE electrophoresis and Western blotting.

Further, in order to confirm the presence of a terminal β1,4-glactose motif which is a human glycan residue, the lectin blotting was carried out using Ricinus communis agglutinin RCA₁₂₀ (Vector Laboratories, USA) which is specific biotinylated lectin, as a probe. Color development was induced using HRP-conjugated streptavidin (KPL Inc., USA). The presence of plant-specific glycan residues β1,2-xylose and α1,3-fucose was confirmed using rabbit anti-HRP-IgG (Sigma, USA) as a probe, and color was developed with HRP-conjugated goat anti-rabbit IgG.

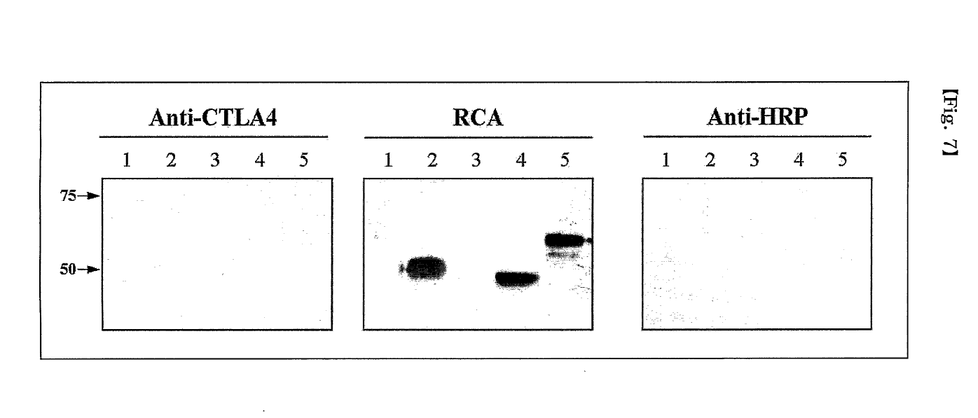

FIG. 7 is a photograph showing Western and Lectin blot patterns of CTLA4Ig following SDS-PAGE electrophoresis thereof. Lane 1: Molecular weight marker, Lane 2: CHO cell-derived CTLA4Ig (CTLA4Ig^{M}), Lane 3: Wild rice cell-derived CTLA4Ig (CTLA4Ig^{P}), Lane 4: Modified molecular weight of rice cell-derived CTLA4Ig having a human glycan structure (CTLA4Ig^{P}-Gal) in accordance with the present invention, and Lane 5: Fetuin as glycoprotein control.

As can be seen from FIG. 7, the modified molecular weight of both CHO cell- and rice cell-derived CTLA4Igs was confirmed to be about 50 kDa.

When it was confirmed using a probe of RCA120 which is lectin specific for a human glycan motif β1,4-glactose, the wild rice cell-derived CTLA4Ig^{P} (Lane 3 of RCA panel in FIG. 7) exhibited no reactivity with RCA120, whereas the rice cell line BR-Os/3D-hGalT or BR-Os/35S-hGalT-derived CTLA4Ig^{P}-Gal having a human glycan motif (Lane 4 of RCA panel) in accordance with the present invention exhibited reactivity with lectin, resulting in color development, similar to animal cell-derived, i.e., CHO cell-derived CTLA4Ig^{M} (Lane 2 of RCA panel).

Further, when rabbit anti-HRP-IgG (Sigma, USA) as a probe was used for assay of plant-specific glycan residues β1,2-xylose and α1,3-fucose, CTLA4Ig^{P}-Gal in accordance with the present invention exhibited significantly low reactivity with lectin, as compared to the wild rice cell-derived counterpart, thus confirming great reduction of the plant-specific glycan residues (Lane 4 of Anti-HRP panel).

### Experimental Example 2

### Half Life of CTLA4Igs In Vivo

In order to ascertain whether an in-vivo half life of the transformed rice cell-derived CTLA4Ig^{P}-Gal having a human glycan structure in accordance with the present invention was increased, CTLA4Ig was intravenously injected into rats and a plasma concentration thereof was measured.

For an intravenous injection of CTLA4Ig, a cannula was surgically inserted into the left vena femoralis of 8-week-old male Sprague-Dawley (SD) rats. After animals were given a recovery period of 2 days, wild rice cell-derived CTLA4Ig^{P} and rice cell line-derived CTLA4Ig^{P}-Gal having a human glycan motif in accordance with the present invention were each diluted in physiological saline and were intravenously injected at a dose of 2 mg/kg to 6 SD rats through the cannula. At various time points of 5 min, 15 min, 30 min, 60 min, 90 min, 150 min, 240 min, 360 min, 480 min, 720 min and 1440 min after intravenous injection of test drugs, blood samples from animals were collected by retro-orbital sinus bleeding using a capillary tube. The blood samples were centrifuged at 3,500 rpm for 10 min, and only the supernatant plasma was separated. Then, a plasma concentration of CTLA4Ig was measured by ELISA.

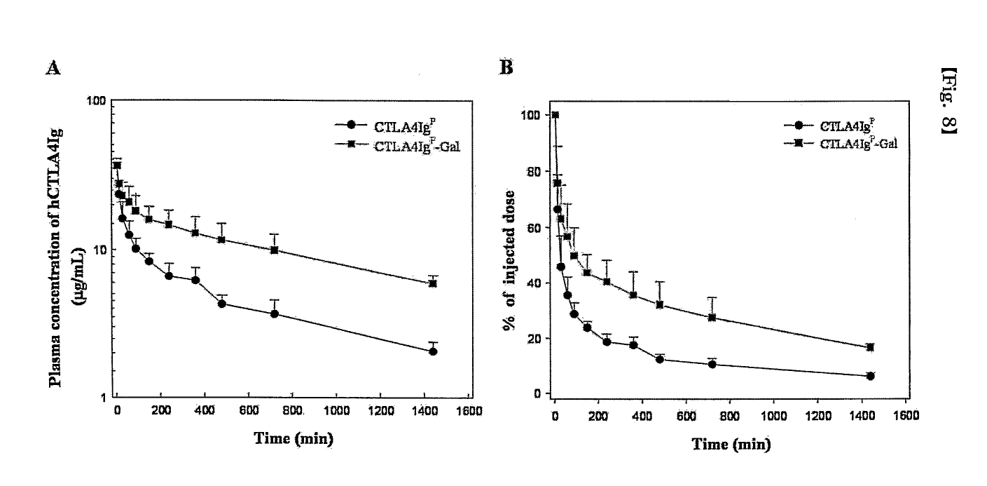

FIG. 8A is a graph showing time-dependent changes of a plasma CTLA4Ig concentration in rats, and FIG. 8B is a graph showing a residual plasma concentration (%) of CTLA4Ig vs a dose of CTLA4Ig. As can be seen from FIGS. 8A and 8B, the wild rice cell-derived CTLA4Ig^{P} (solid circle) exhibited a rapid decrease of the plasma CTLA4Ig concentration after intravenous administration thereof, i.e., up to a 50% decrease within 30 min and a decrease to a 10% level of the initial concentration 24 hours later. On the other hand, the transformed rice cell-derived CTLA4Ig^{P}-Gal (solid square) in accordance with the present invention exhibited a slow decrease of the plasma concentration, maintaining more than 40% level of the initial concentration up to 6 hours, and more than 20% level of the initial concentration even after 24 hours. Table 1 below shows pharmacokinetic parametric values of the CTLA4Ig^{P} fusion protein calculated from the plasma concentration data of rats. As can be seen from Table 1, the transformed rice cell-derived CTLA4Ig^{P}-Gal having a human glycan structure in accordance with the present invention exhibited a decrease of the blood clearance rate (CL_{T}) to a 1/3 level of that of the wild rice cell-derived CTLA4Ig^{P}, thus confirming that both a half life (t1/2) and a mean residence time (MLT) of the desired fusion protein were increased.

TABLE 1

| | AUC_{inf} | t_{1/2(α)} | t_{1/2(β)} | Cₘₐₓ | CL_{T} | MRT | Vdₛₛ |
|---|---|---|---|---|---|---|---|
| Treatment | (min · μg/mL) | (min) | (min) | (μg/mL) | (mL/min) | (min) | (mL) |
| CTLA4Ig^{P} | 7222.2 (1728.9) | 12.7 (8.0) | 414.3 (89.1) | 45.0 (5.8) | 0.09 (0.02) | 549.8 (113.6) | 46.3 (6.8) |
| CTLA4Ig^{P}-Gal | 21291.2 (5698.3) | 15.6 (7.3) | 767.1 (142.3) | 42.7 (7.4) | 0.03 (0.01) | 1083.1 (204.0) | 31.7 (6.6) |

The above results represent that the transformed rice cell-derived CTLA4Ig^{P}-Gal of the present invention can overcome the problems associated with rapid lowering of the plasma concentration suffered by the wild rice cell-derived CTLA4Ig^{P}, due to having a human glycan structure. Such enhancing effects of an in vivo half life imply that it is possible to improve efficacy of the plant cell-derived recombinant CTLA4Ig as a pharmaceutical.

### Experimental Example 3

### Immunosuppressive Activity of CTLA4Igs

In order to examine whether the transformed rice cell-derived CTLA4Ig^{P}-Gal having a human glycan structure in accordance with the present invention has an immunosuppressive activity, an in vitro activity test was carried out using mouse splenocytes.

In order to determine T-cell proliferative capacity, the spleen was excised from BDFI mice and splenocytes were harvested therefrom. A cell concentration was adjusted to 1×10⁶ cells/mL and 200 μl/well was aliquoted and incubated on a 96-well plate. Samples to test an immunosuppressive activity were added thereto. This was followed by addition of 2 μg/mL of ConA, a mitogen that induces proliferation of T lymphocytes, and incubation in a 5% CO₂ incubator at 37° C. for 3 days. 18 hours prior to the completion of incubation, 1 μCi/well of [³H]-thymidine was added and cells were harvested from each well using an automatic cell harvester. Then, the degree of immunocyte proliferation was examined by measuring the degree of incorporation of [³H]-thymidine into DNA. In this manner, a degree of inhibition of ConA-induced T lymphocyte proliferation by CTLA4Ig was measured.

Further, effects of CTLA4Ig were also examined on the production of T cell-derived cytokines. Mouse splenocytes (1×10⁶ cell/mL) were plated and then treated with the rice cell-derived CTLA4Ig^{P} protein or animal cell-derived CTLA4Ig^{M} protein together with ConA (1 μg/mL). 24 hours later, the culture sampling was carried out. In order to measure concentrations of cytokines responsible for immune response in the culture medium, capture antibodies for individual cytokines were appropriately diluted in PBS and aliquoted at a concentration of 100 μl/well into an ELISA plate. The ELISA plate was sealed, followed by overnight reaction at 4° C. to complete antibody coating. The antibody-coated plate was washed three times with phosphate buffer (PBS), and 200 μl/well of a blocking solution (1% BSA, 5% sucrose in PBS) was added to the plate, followed by reaction at room temperature for 1 hour. After the plate was washed three times again with phosphate buffer, the culture samples and reference standards for cytokines were diluted and aliquoted into the capture antibody-coated plate, followed by reaction for 2 hours. Again, the plate was washed three times with phosphate buffer and 100 μl/well of biotinylated detection antibodies were aliquoted to the plate, followed by reaction for 2 hours. The plate was washed and 100 μl/well of HRP labeled streptavidin (KPL, USA) was added to the plate, followed by reaction at room temperature for 20 min. Finally, the plate was washed with phosphate buffer and a substrate solution was added thereto. When color was appropriately developed, a stop solution was added to terminate the reaction and an absorbance was measured at 450 nm.

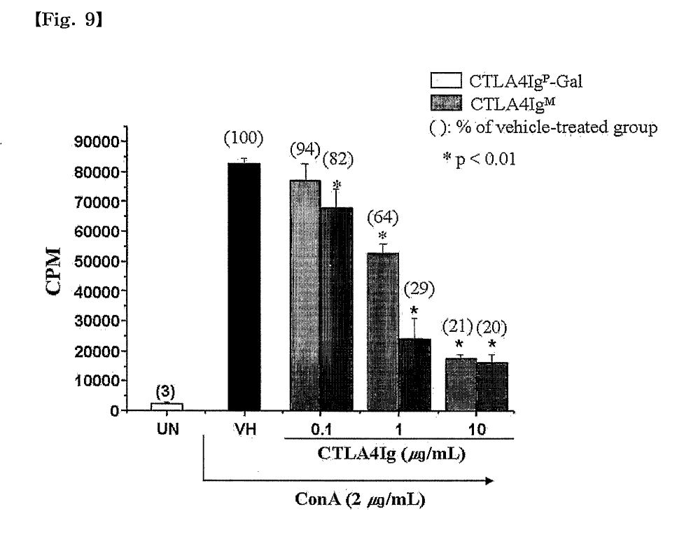

FIG. 9 graphically shows the results of an immunosuppressive activity test by confirming effects of rice cell-derived CTLA4Ig^{P}-Gal having a human glycan structure on the proliferation of T cells induced by ConA (2 μg/mL). As can be seen from FIG. 9, in comparison with a buffer-treated (vehicle) group (VH) as a negative control, CHO cell-derived CTLA4Ig^{M} as a positive control and rice cell-derived CTLA4Ig^{P}-Gal of the present invention inhibited T cell proliferation and exhibited comparable immunosuppressive activity. Both samples showed inhibitory effects at a concentration starting from 0.1 μg/mL, inhibited the activation of T cells in a concentration-dependent manner, and exhibited antiproliferative effects of about 80% at a concentration of 10 μg/mL, as compared to the control group.

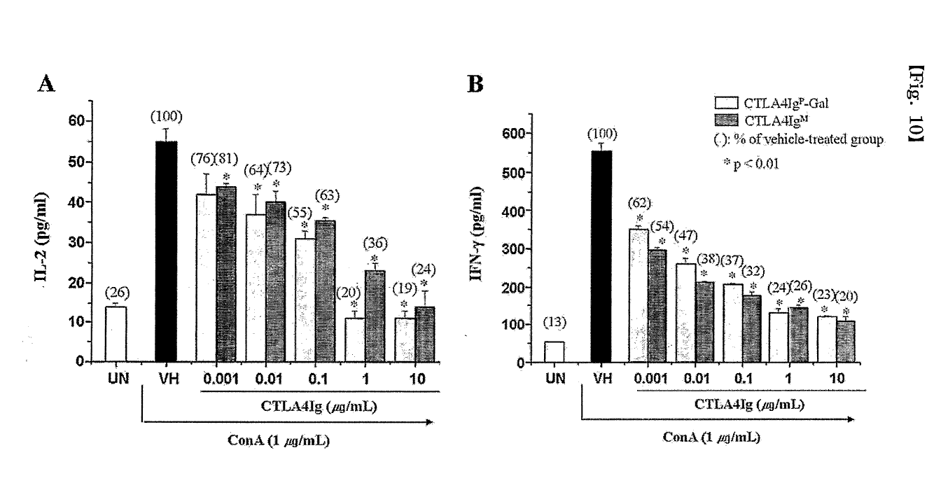

FIG. 10 graphically shows effects of rice cell-derived CTLA4Ig^{P}-Gal having a human glycan structure in accordance with the present invention on IL-2 and IFN-γ which are major cytokines secreted from T cells. When mouse splenocytes were treated with CTLA4Ig^{P}-Gal, production of IL-2 induced by ConA (1 μg/mL) was significantly decreased (FIG. 10A). When CTLA4Ig^{P} was administered at a concentration of more than 0.001 μg/mL, decreased production of IL-2 was statistically significant (p<0.01). At a concentration of 10 μg/mL, inhibitory effects of 81% were obtained. Further, both CTLA4Ig^{P}-Gal and CTLA4Ig^{M} also exhibited inhibitory effects on IFN-γ, similar to IL-2. In addition, inhibitory capacity was also similar between two sample groups (FIG. 10B). Both of two samples showed statistically significant (p<0.01) inhibitory effects at a concentration of 0.001 μg/mL or higher, and showed inhibitory effects of 77% and 80% at a dose of 10 μg/mL, as compared to a negative (vehicle) control (VH).

As can be seen from the results of Experimental Example as above, CHO-derived CTLA4Ig^{M} and the rice cell-derived CTLA4Ig^{P}-Gal having a human glycan structure in accordance with the present invention inhibited a variety of T cell-mediated immune activities. Further, the inventive rice cell-derived CTLA4Ig^{P}-Gal exhibited pronounced immunosuppressive activity equal to or higher than CHO-derived CTLA4Ig^{M}, in suppression of immune activities of T cells. Therefore, these results suggest that the rice cell-derived CTLA4Ig^{P}-Gal having a human glycan structure in accordance with the present invention can be probably developed as therapeutics for a variety of immune diseases where T cells play an important role.

### INDUSTRIAL APPLICABILITY

The present invention provides a recombinant vector pBI-3D-hGalT or pBI-35S-hGalT containing a human β1,4-galactosyltransferase gene; a cell line transformed with a recombinant vector pMYN414 containing a cytotoxic T-lymphocyte antigen 4-immunoglobulin (CTLA4Ig) fusion protein gene and the recombinant vector pBI-3D-hGalT or pBI-35S-hGalT; and a method for producing a plant-derived recombinant human CTLA4Ig (CTLA4Ig^{P}) fusion protein with a human glycan structure using the same. The plant cell-derived CTLA4Ig fusion protein, which has a human glycan structure and is produced according to the present invention, exhibits an improved in vivo half life as compared to conventional plant-derived proteins, due to the presence of a human-like glycan structure. Consequently, the present invention using the plant expression system enables low-cost mass production of a CTLA4Ig^{P} fusion protein having an immunosuppressive activity comparable to that of the CTLA4Ig^{M} fusion protein expressed in conventional animal cell expression systems.

What is claimed is:

1. A recombinant vector pBI-3D-hGalT containing a human β1,4-galactosyltransferase (hGalT) gene and having a cleavage map as shown in FIG. 1.

2. The recombinant vector pBI-3D-hGalT of claim 1, wherein the hGalT gene has a nucleotide sequence as set forth in SEQ ID NO: 1.

3. A recombinant vector pBI-35S-hGalT containing a human β1,4-galactosyltransferase (hGalT) gene and having a cleavage map as shown in FIG. 2.

4. The recombinant vector pBI-35S-hGalT of claim 3, wherein the hGalT gene has a nucleotide sequence as set forth in SEQ ID NO: 1.

5. A plant cell transformed with a recombinant vector pMYN414 containing a human cytotoxic T lymphocyte antigen 4-immunoglobulin (CTLA4Ig) fusion protein gene and having a cleavage map as shown in FIG. 3 and the recombinant vector pBI-3D-hGalT of claim 1.

6. The plant cell of claim 5, wherein the CTLA4Ig fusion protein gene has a nucleotide sequence as set forth in SEQ ID NO: 2.

7. The plant cell of claim 5, wherein the plant cell is any one selected from the group consisting of rice (Oryza sativa L.), tobacco (Nicotiana tabacum), maize (Zea mays), soybean (Glycine max), wheat (Triticum aestivum), tomato (Lycopersicon esculentum), rape (Brassica napus) and potato (Solanum tuberosum).

8. The plant cell of claim 7, wherein the plant cell is Oryza sativa L.

9. The plant cell of claim 8, wherein the plant cell is Oryza saliva L. under Accession Number KCTC 11141 BP.

10. A plant cell transformed with a recombinant vector pMYN414 containing a human cytotoxic T lymphocyte antigen 4-immunoglobulin (CTLA4Ig) fusion protein gene and having a cleavage map as shown in FIG. 3 and the recombinant vector pBI-35S-hGalT of claim 3.

11. The plant cell of claim 10, wherein the CTLA4Ig fusion protein gene has a nucleotide sequence as set forth in SEQ ID NO: 2.

12. The plant cell of claim 10, wherein the plant cell is any one selected from the group consisting of rice (Oryza sativa L.), tobacco (Nicotiana tabacum), maize (Zea mays), soybean (Glycine max), wheat (Triticum aestivum), tomato (Lycopersicon esculentum), rape (Brassica napus) and potato (Solanum tuberosum).

13. The plant cell of claim 12, wherein the plant cell is Oryza sativa L.

14. The plant cell of claim 13, wherein the plant cell is Oryza sativa L. under Accession Number KCTC 11142BP.

15. A method for producing a plant-derived recombinant human CTLA4Ig (CTLA4Ig^{P}) fusion protein comprising suspension-culturing the transformed plant cell of claim 5 and separating CTLA4Ig^{P} from the culture medium.

16. The method of claim 15, wherein the suspension-culturing is carried out in a medium containing sugars, growth regulators and antibiotics for selection.

17. A method for producing a plant-derived recombinant human CTLA4Ig (CTLA4Ig^{P}) fusion protein comprising suspension-culturing the transformed plant cell of claim 10 and separating CTLA4Ig^{P} from the culture medium.

18. The method of claim 17, wherein the suspension-culturing is carried out in a medium containing sugar, growth regulators and antibiotics for selection.

19. A plant-derived recombinant human CTLA4Ig (CTLA4Ig^{P}) fusion protein isolated and produced from a suspension culture of the transformed plant cell of claim 5.

20. A plant-derived recombinant human CTLA4Ig (CTLA4Ig^{P}) fusion protein isolated and produced from a suspension culture of the transformed plant cell of claim 10.

21. An immunosuppressive pharmaceutical composition comprising the CTLA4Ig^{P} fusion protein of claim 19 as an active ingredient.

22. An immunosuppressive pharmaceutical composition comprising the CTLA4Ig^{P} fusion protein of claim 20 as an active ingredient.

23. A use of the CTLA4Ig^{P} fusion protein of claim 19 for the preparation of an immunosuppressant.

24. A use of the CTLA4Ig^{P} fusion protein of claim 20 for the preparation of an immunosuppressant.

25. A method for inhibiting an immune response, comprising administering to a mammal a therapeutically effective amount of the CTLA4Ig^{P} fusion protein of claim 19.

26. A method for inhibiting an immune response, comprising administering to a mammal a therapeutically effective amount of the CTLA4Ig^{P} fusion protein of claim 20.


SEQUENCE LISTING (8 sequences)

SEQ ID NO: 1 - DNA, 1189 bp, Artificial Sequence
  hGalT
aaatctagagcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctactagtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagcgttttggtacacggtacc

SEQ ID NO: 2 - DNA, 1167 bp, Artificial Sequence
  CTLA4Ig
atgcaggtgctgaacaccatggtgaacaaacacttcttgtccctttcggtcctcatcgtcctccttggcctctcctccaacttgacagccggggcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - DNA, 29 bp, Artificial Sequence
  forward primer GalS1
aaatctagagcgatgccaggcgcgtccct

SEQ ID NO: 4 - DNA, 29 bp, Artificial Sequence
  reverse primer GalS2
aatactagtagcggggactcctcagggca

SEQ ID NO: 5 - DNA, 27 bp, Artificial Sequence
  forward primer Gal1S
aagactagtgggccccatgctgattga

SEQ ID NO: 6 - DNA, 28 bp, Artificial Sequence
  reverse primer Gal2K
gtaggtaccgtgtaccaaaacgctagct

SEQ ID NO: 7 - DNA, 41 bp, Artificial Sequence
  forward primer CTLA4-F1
tccaacttgacagccggggcaatgcacgtggcccagcctgc

SEQ ID NO: 8 - DNA, 26 bp, Artificial Sequence
  reverse primer CTLA4-R1
ctctgcagaatctgggcacggttctg